(19) United States
(12) Patent Application Publication
(10) Pub. No.: US 2009/0149673 A1
(43) Pub. Date: Jun. 11, 2009

(54) SYNTHETIC NON-FOULING AMINO ACIDS

(75) Inventors: Zheng Zhang, Cambridge, MA (US); William Shannan O'Shaughnessey, Boston, MA (US); Michael Hencke, Cambridge, MA (US); Trevor Squier, Peabody, MA (US); Christopher R. Loose, Cambridge, MA (US)

Correspondence Address: Pabst Patent Group LLP, 1545 PEACHTREE STREET NE, SUITE 320, ATLANTA, GA, 30309, US

(73) Assignee: Semprus Biosciences Corp.

(21) Appl. No.: 12/329,360

(22) Filed: Dec. 5, 2008

Related U.S. Application Data
(60) Provisional application No. 61/026,340, filed on Feb. 5, 2008.
(60) Provisional application No. 60/992,629, filed on Dec. 5, 2007.

(51) Int. Cl. C07C 229/06

(52) U.S. Cl. 562/553

(57) ABSTRACT

Synthetic amino acids containing one or more non-fouling groups or moieties are described herein. In one embodiment, the amino acid has the following chemical formula:

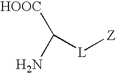

where L is a linker group and Z is a non-fouling group including, but not limited to, polyethylene glycol (PEG); oligoethylene glycol (OEG); zwitterionic group, such as phosphorycholine, carboxybetaine, and sulfobetaine; groups that are hydrogen bond acceptors but not hydrogen bond donors. The non-fouling amino acids can be incorporated into a bioactive peptide as single amino acid residues, multiples amino acid residues, or as blocks of amino acids. The non-fouling amino acids, or peptides containing one or more non-fouling amino acids, can be applied to surfaces in order to improve biocompatibility, reduce thrombogenesis, and/or reduce fouling by proteins or bacteria present in solution.

### CROSS-REFERENCE TO RELATED APPLICATIONS

This application claims benefit of U.S. Ser. No. 61/026,340, entitled “Synthetic Non-Fouling Amino Acids” by Zheng Zhang, William Shannan O'Shaughnessey, Christopher R. Loose and Michael Hencke, filed in the U.S. Patent and Trademark Office on Feb. 5, 2008, which is incorporated by referenced in its entirety.

### FIELD OF THE INVENTION

The present invention is in the field of amino acids which resist non-specific protein adsorption or cellular adhesion and bioactive peptides prepared from such amino acids.

### BACKGROUND OF THE INVENTION

The success of a material, coating or device intended for biomedical use depends on numerous factors, one of which is resistance to fouling. Devices that are susceptible to fouling often exhibit significantly reduced function over time when used in vivo and have been implicated in thrombus formation, increased cases of infection and in some instances, catastrophic device failure. Materials and coatings, with a high resistance to fouling have been developed for biomedical use, but these materials suffer from a short in vivo lifetime due to degradation of the material or surface. Other systems have been developed to take advantage of drug elution as a method to prevent fouling of a device. Unfortunately the non-fouling performance of the device is limited to the duration of the drug release. Other classes of molecules and materials must be developed to address these issues of in vivo stability and lifetime.

Zwitterion coated surfaces and materials comprising zwitterionic moieties have been shown to exhibit fouling resistance that outperforms many of the current materials and devices currently on the market. Zwitterionic surfaces have been shown to resist or actively prevent device related thrombus formation when applied in vivo. Zwitterionic moieties have also been shown to prevent attachment of bacteria to the surface on which they are attached. Furthermore the bacteriostatic properties of the zwitterionic moiety can work in tandem with immobilized antimicrobial agents to create surfaces highly resistant to bacterial biofilm formation. Other bioactive agents, such as antithrombotic, anti-inflamatories, or cell signaling agents can also be beneficial to device performance when tethered to the device surface. However, as with antimicrobials, fouling of the device may mask these tethered agents and prevent interaction with their desired targets.

While many standard polymerization approaches have been explored to add non-fouling materials to a substrate prior to bioactive agent immobilization, an alternate approach is to design zwitterionic materials or surface coatings where the zwitterionic moiety is part of an amino acid. Proteins or peptides can then have this non-fouling functionality integrated within their structure during synthesis. In addition, by altering zwitterionic amino acid stereochemistry, from 1 to d, the in vivo half life of materials and coatings comprising of said amino acid could be tailored to degrade or remain stable over a wide range of time in vivo. Such zwitterionic amino acids would also provide a flexible platform for the design of novel surface coatings and materials that would resist biofouling and non-specific protein adsorption. Zwitterionic amino acids could not only be used to create novel, non-fouling materials and coatings with considerable stability, but could also be used to prevent the in vivo degradation of other peptide and protein therapeutics. Stabilization of said peptides and proteins would open the door for therapeutic applications that were previously made impossible by the quick in vivo degradation of such molecules.

It is therefore an object of the present invention to provide synthetic amino acids which are non-fouling, methods of making thereof, and bioactive peptides containing such amino acids.

It is further an object of the invention to provide non-fouling peptides that exhibit bioactivity, such as antimicrobial activity, anti-thrombogenesis properties, and/or biomarker properties and methods of making and use thereof.

It is another object of the invention to provide amino acids which can be used in peptides and proteins to decrease uptake by the reticuloendothelial system (RES), decrease the rate of enzymatic degradation, or decrease the rate of clearance from the body by other mechanisms, and thereby provide longer half-lives.

### SUMMARY OF THE INVENTION

Synthetic amino acids containing one or more non-fouling groups or moieties are described herein. In one embodiment, the amino acid has the following chemical formula:

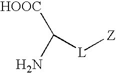

where L is a linker group and Z is a non-fouling group including, but not limited to, polyethylene glycol (PEG); oligoethylene glycol (OEG); zwitterionic group, such as phosphorycholine, carboxybetaine, and sulfobetaine; groups that are hydrogen bond acceptors but not hydrogen bond donors, such as amides, amide derivatives, amines, amine derivatives, cyclic ethers, sugar derivatives, sulfonates, carboxylic acids, carboxylic acid derivatives, and nitrites; and combinations thereof. Protecting groups including F-moc, Boc, and non-fouling groups may also be attached to the C-terminus of the amino acid, the N-terminus of the amino acid, and/or the linker group to enable traditional chemical synthesis without altering specific desired moieties. The non-fouling group, Z, may also be protected using protecting groups such as F-moc and Boc. Amino acids can be natural or non-natural, D or L amino acids.

The non-fouling amino acids can be incorporated into a bioactive peptide as single amino acid residues, multiples amino acid residues, or as blocks of amino acids. The non-fouling amino acids can be incorporated into the peptides randomly or in a specific sequence. For example, a peptide can be designed to have an adhesive segment for attaching the peptide to a surface, a non-fouling segment, and a bioactive segment. In one embodiment, the bioactive segment is an antimicrobial peptide. In another embodiment, the bioactive segment is a peptide that has anti-thrombogenesis properties. In still another embodiment, the bioactive segment is a biomarker, cell adhesion peptide such as RGD, bone morphogenic protein mimetics, or other bioactive peptide.

These non-natural amino acids may be incorporated into peptides or proteins during chemical synthesis using protected versions of the amino acid including, but not limited to, F-moc, Boc, or Z groups. Depending on the chemical nature of the side chain, additional protecting groups may be necessary to block the sidechain from participating in the peptide synthesis reaction. Alternatively, these non-fouling, non-natural amino acids can be incorporated recombinantly by changing the t-RNA assigned to one codon to incorporate the amino acid. The altered genetic code could be used through in vitro translation or through modifying the code of a host production organism. The host organism can be eukaryotic or prokaryotic.

Alternatively, the zwitterionic amino acid or peptide can be synthesized through a post-reaction of an amino acid or peptide possessing amine groups. In one embodiment, a sulfoboxybetaine amino acid or peptide can be synthesized from the reaction using an N,N-dimethyl amino group and a propane sultone. In another embodiment, a carboxybetaine amino acid or peptide can be synthesized from the reaction using an N,N-dimethyl amino group and a propiolactone. In a third embodiment, a carboxybetaine amino acid or peoptide can be synthesized using a tertiary amino group and a bromoester.

The non-fouling amino acids, or peptides containing one or more non-fouling amino acids, can be applied to surfaces, particularly the surfaces of medical devices, in order to improve biocompatibility, reduce thrombogenesis (such as on the surface of stents), and reduce fouling by proteins or bacteria present in solution. This is particularly applicable for surfaces where immobilized proteins and peptides are present because non-specific protein or cell fouling may cover these immobilized molecules. Immobilized protein or peptide surfaces may be used in arrays/sensors, or to create antimicrobial surfaces using antimicrobial peptides.

In addition, non-fouling groups may be incorporated into molecules, particularly proteins or peptides, in solution to improve stability or half-life in the blood stream. The molecules can be incorporated into a pharmaceutically acceptable carrier to form pharmaceutical compositions. The compositions can be administered enterally or parenterally. In a preferred embodiment, the compositions are administered parenterally.

### DETAILED DESCRIPTION OF THE INVENTION

### I. Definitions

“Amino acid residue” and “peptide residue”, as used herein, refer to an amino acid or peptide molecule without the —OH of its carboxyl group (C-terminally linked) or one proton of its amino group (N-terminally linked). In general the abbreviations used herein for designating the amino acids and the protective groups are based on recommendations of the IUPAC-IUB Commission on Biochemical Nomenclature (see Biochemistry (1972) 11:1726-1732). Amino acid residues in peptides are abbreviated as follows: Alanine is Ala or A; Cysteine is Cys or C; Aspartic Acid is Asp or D; Glutamic Acid is Glu or E; Phenylalanine is Phe or F; Glycine is Gly or G; Histidine is His or H; Isoleucine is Ile or I; Lysine is Lys or K; Leucine is Leu or L; Methionine is Met or M; Asparagine is Asn or N; Proline is Pro or P; Glutamine is Gln or Q; Arginine is Arg or R; Serine is Ser or S; Threonine is Thr or T; Valine is Val or V; Tryptophan is Trp or W; and Tyrosine is Tyr or Y. Formylmethionine is abbreviated as fMet or Fm. By the term “residue” is meant a radical derived from the corresponding ÿ-amino acid by eliminating the OH portion of the carboxyl group and one of the protons of the ÿ-amino group. The term “amino acid side chain” is that part of an amino acid exclusive of the CH(NH₂)COOH backbone, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂ CH₃ (the side chain of isoleucine), —CH₂CH(CH₃)₂ (the side chain of leucine) or —H (the side chain of glycine).

“Non-naturally occurring amino acid”, as used herein, refers to any amino acid that is not found in nature. Non-natural amino acids include any D-amino acids, amino acids with side chains that are not found in nature, and peptidomimetics. Examples of peptidomimetics include, but are not limited to, b-peptides, g-peptides, and d-peptides; oligomers having backbones which can adopt helical or sheet conformations, such as compounds having backbones utilizing bipyridine segments, compounds having backbones utilizing solvophobic interactions, compounds having backbones utilizing side chain interactions, compounds having backbones utilizing hydrogen bonding interactions, and compounds having backbones utilizing metal coordination. * All of the amino acids in the human body, except glycine, exist as the D and L forms. Nearly all of the amino acids occurring in nature are the L-forms. D-forms of the amino acids are not found in the proteins of higher organisms, but are present in some lower forms of life, such as in the cell walls of bacteria. They also are found in some antibiotics, among them, streptomycin, actinomycin, bacitracin, and tetracycline. These antibiotics can kill bacterial cells by interfering with the formation of proteins necessary for viability and reproduction. Non-naturally occurring amino acids also include residues, which have side chains that resist non-specific protein adsorption, which may be designed to enhance the presentation of the antimicrobial peptide in biological fluids, and/or polymerizable side chains, which enable the synthesis of polymer brushes using the non-natural amino acid residues within the peptides as monomeric units.

“Polypeptide”, “peptide”, and “oligopeptide” encompasses organic compounds composed of amino acids, whether natural, synthetic or mixtures thereof, that are linked together chemically by peptide bonds. Peptides typically contain 3 or more amino acids, preferably more than 9 and less than 150, more preferably less than 100, and most preferably between 9 and 51 amino acids. The polypeptides can be “exogenous,” or “heterologous,” i.e. production of peptides within an organism or cell that are not native to that organism or cell, such as human polypeptide produced by a bacterial cell, Exogenous also refers to substances that are not native to the cells and are added to the cells, as compared to endogenous materials, which are produced by the cells. The peptide bond involves a single covalent link between the carboxyl group (oxygen-bearing carbon) of one amino acid and the amino nitrogen of a second amino acid. Small peptides with fewer than about ten constituent amino acids are typically called oligopeptides, and peptides with more than ten amino acids are termed polypeptides. Compounds with molecular weights of more than 10,000 Daltons (50-100 amino acids) are usually termed proteins.

“Antimicrobial” as used herein, refers to molecules that kill (i.e., bactericidal) or inhibit the growth of (i.e., bacteristatic) microorganisms including bacteria, yeast, fungi, mycoplasma, viruses or virus infected cells, cancerous cells, and/or protozoa.

“Antimicrobial peptide” (“AmP”), as used herein, refers to oligopeptides, polypeptides, or peptidomimetics that kill (i.e., are bactericidal) or inhibit the growth of (i.e., are bacteristatic) microorganisms including bacteria, yeast, fungi, mycoplasma, viruses or virus infected cells, and/or protozoa. Generally, antimicrobial peptides are cationic molecules with spatially separated hydrophobic and charged regions. Exemplary antimicrobial peptides include linear peptides that form an a-helical structure in membranes or peptides that form β-sheet structures, optionally stabilized with disulfide bridges in membranes. Representative antimicrobial peptides include, but are not limited to, cathelicidins, defensins, dermcidin, and more specifically magainin 2, protegrin, protegrin-1, melittin, 11-37, dermaseptin 01, cecropin, caerin, ovispirin, cecropin A melittin hybrid, and alamethicin, or hybrids or analogues of other AmPs. Naturally occurring antimicrobial peptides include peptides from vertebrates and non-vertebrates, including plants, humans, fungi, microbes, and insects.

“Adhesion”, as used herein, refers to the non-covalent or covalent attachment of a protein, cell, or other substance to a surface. The amount of adhered substance may be quantified by sonicating and/or rinsing the surface with an appropriate resuspension agent such as Tween or SDS, and quantifying the amount of substance resuspended.

“Bioactive agent” or “active agent” or “biomolecule”, used here synonymously, refers to any organic or inorganic therapeutic, prophylactic or diagnostic agent that actively or passively influences a biological system. For example, a bioactive agent can be an amino acid, antimicrobial peptide, immunoglobulin, an activating, signaling or signal amplifying molecule, including, but not limited to, a protein kinase, a cytokine, a chemokine, an interferon, tumor necrosis factor, growth factor, growth factor inhibitor, hormone, enzyme, receptor-targeting ligand, gene silencing agent, ambisense, antisense, an RNA, a living cell, cohesin, laminin, fibronectin, fibrinogen, osteocalcin, osteopontin, or osteoprotegerin. Bioactive agents can be proteins, glycoproteins, peptides, oligliopeptides, polypeptides, inorganic compounds, organometallic compounds, organic compounds or any synthetic or natural, chemical or biological compound.

“Non-fouling”, as used herein, means that the composition reduces or prevents the amount of adhesion of proteins, including blood proteins, plasma, cells, tissue and/or microbes to the substrate relative to the amount of adhesion to a reference polymer such as polyurethane. Preferably, a device surface will be substantially non-fouling in the presence of human blood. Preferably the amount of adhesion will be decreased 20%, 50%, 75%, 90%, 95%, or most preferably 99%, 99.5%, 99.9% relative to the reference polymer. Non-fouling activity with respect to protein, also referred to as “non-specific protein adsorption resistance” may be measured using an ELISA assay. For solutions containing only a single protein, protein adsorption can be measured by ELISA assay. The sample is first incubated in the protein solution, then rinsed to remove loosely adhered proteins. It is then exposed to a solution containing a calorimetrically labeled antigen to the specific protein and once again rinsed to remove loosely adhered material. Finally, the substrate is treated with solution to remove the antigen and the concentration of the antigen measured by UV-Vis spectroscopy. For mixed protein solutions, such as whole plasma, surface plasmon resonance (SPR) or optical waveguide lightmode spectroscopy (OWLS) can be utilized to measure surface protein adsorption without necessitating the use of individual antigens for each protein present in solution. Additionally, radiolabeled proteins may be quantified on the surface after adsorption from either one protein or complex mixtures. Non-fouling activity with respect to bacteria may be quantified by exposing treated substrates (and untreated controls) to between 1×10⁵-10⁷ CFU/ml of a given organism suspended in PBS or more complex media for 2 hours. The samples are then rinsed to remove loosely adherent cells, placed in fresh PBS, and then sonicated to re-suspend the adherent bacteria in solution. Serial dilutions of this supernatant solution can then be made, plated, and grown up over night to provide a quantitative measure of bacterial adhesion on the treated sample versus the control. Preferably at least a 1, 2, 3 or 4 log reduction in bacterial count occurs relative to colonization on a control. Similar adherence assays are known in the art for assessing platelet, cell, or other material adhesion to the surface.

“Biocompatibility” is the ability of a material to perform with an appropriate host response in a specific situation (Williams, D. F. Definitions in Biomaterials. In: Proceedings of a consensus Conference of the European Society for Biomaterials. Elsevier: Amsterdam, 1987).

“Biological fluids” are fluids produced by organisms containing proteins and/or cells, as well as fluids and excretions from microbes. This includes, but is not limited to, blood, saliva, urine, cerebrospinal fluid, tears, semen, and lymph, or any derivative thereof (e.g., serum, plasma).

“Brush”, or “Polymer Brush” as used herein synonymously, refers to a relatively high density of polymer chains stretched away from the polymer or polymers due to the volume-excluded effect. The polymer-chains are typically end-tethered to the substrate. In mixed brushes, two or more different polymers grafted to the same substrate constitute the brush.

“Branch” and “Branched tether,” are used interchangeably and refer to a polymer structure which originates from a single polymer chain but terminates in two or more polymer chains. The polymer in question may be a homopolymer or multicomponent copolymer. Branched tether polymer structure may be ordered or random, may be composed, in whole or in part, of non-fouling material, and may be utilized to immobilize one or more molecules of one or more bioactive agents. In one embodiment the branched tether is a dendrimer. A branched tether may be immobilized directly to a substrate or to a coating covering a substrate.

“Coupling agent”, as used herein, refers to any molecule or chemical substance which activates a chemical moiety, either on the bioactive agent or on the material to which it will be attached, to allow for formation of a covalent or non-covalent bond between the bioactive agent wherein the material does not remaining in the final composition after attachment.

“Cysteine”, as used herein, refers to the amino acid cysteine or a synthetic analogue thereof, wherein the analogue contains a free sulfhydryl group.

“Degradation products” are atoms, radicals, cations, anions, or molecules which are derived from a bioactive agent or composition and which are formed as the result of hydrolytic, oxidative, enzymatic, or other chemical processes over the course of 14, 30, 120, 365, or 1000 days.

“Density”, as used herein, refers to the mass of material, which without limitation may include non-fouling materials or bioactive agents, that is immobilized per surface area of substrate.

“Effective surface density”, as used herein, means the range of densities suitable to achieve an intended surface effect, which without limitation may be antimicrobial or non-fouling effect.

“Hydrophilic” refers to polymers, materials, or functional groups which generally associate with water. These materials include without limitation materials with hydroxyl, zwitterionic, carboxy, amino, amide, phosphate, hydrogen bond formers, and ether.

“Immobilization” or “immobilized”, as used herein, refers to a material or bioactive agent that is covalently attached directly or indirectly to a substrate. “Co-immobilization” refers to immobilization of two or more agents.

“In vivo stability” refers to materials which are not degraded in organism over a defined period of time.

“Non-degradable” or “stable”, as used herein synonymously, refers to material compositions that do not react within a biological environment either hydrolytically, reductively, enzymatically or oxidatively to cleave into smaller pieces. Preferably non-degradable materials retain >25%, >50%, >75%, >90%, >95%,or >99% of their original material properties such as surface contact angle, non-fouling, and/or bactericidal activity for a time of 7, 14, 30, 120, 365, or 1000 days in media, serum, or in vivo.

“Substrate”, as used herein, refers to the material on which a non-fouling coating is applied, or which is formed all or in part of non-fouling material, or on which the non-fouling and/or anti-microbial agents are immobilized.

“Membrane-targeting antimicrobial agent”, as used herein, refers to any antimicrobial agent that retains its bactericidal or bacteriostatic activity when immobilized on a substrate and can therefore be used to create an immobilized antimicrobial surface. In one embodiment, the membrane-targeting antimicrobial agent is an antimicrobial peptide, and in another embodiment it is a quaternary ammonium compound or polymer. “Immobilized bactericidal activity” as used herein, refers to the reduction in viable microorganisms including bacteria, yeast, fungi, mycoplasma, viruses or virus infected cells, and/or protozoa that contact the surface. For bacterial targets, bactericidal activity may be quantified as the reduction of viable bacteria based on the ASTM 2149 assay for immobilized antimicrobials, which may be scaled down for small samples as follows: an overnight culture of a target bacteria in a growth medium such as Cation Adjusted Mueller Hinton Broth, is diluted to approximately 1×10⁵ cfu/ml in pH 7.4 Phosphate Buffered Saline using a predetermined calibration between OD600 and cell density. A 0.5 cm² sample of immobilized antimicrobial surface is added to 0.75 ml of the bacterial suspension. The sample should be covered by the liquid and should be incubated at 37° C. with a sufficient amount of mixing that the solid surface is seen to rotate through the liquid. After 1 hour of incubation, serial dilutions of the bacterial suspension are plated on agar plates and allowed to grow overnight for quantifying the viable cell concentration. Preferably at least a 1, 2, 3 or 4 log reduction in bacterial count occurs relative to a control of bacteria in phosphate buffered saline (PBS) without a solid sample.

“Coating”, as used herein, refers to any temporary, semi-permanent or permanent layer, or layers, treating or covering a surface. The coating may be a chemical modification of the underlying substrate or may involve the addition of new materials to the surface of the substrate. It includes any increase in thickness to the substrate or change in surface chemical composition of the substrate. A coating can be a gas, vapor, liquid, paste, semi-solid or solid. In addition, a coating can be applied as a liquid and solidified into a solid coating.

“Undercoating,” as used herein, refers to any coating, combination of coatings, or functionalized layer covering an entire substrate surface or a portion thereof under an additional coating. In one embodiment, the undercoating is used to alter the properties of one or more subsequent coatings or layers.

“Top coating,” as used herein, refers to any coating, combination of coatings, or functionalized layer applied on top of a undercoating, another top coating or directly to a substrate surface. A top coating may or may not be the final coating applied to a substrate surface. In one embodiment a top coat is covalently attached to an undercoating. In another embodiment a top coating is encapsulated in a protective coating, which helps extend the top coatings storage life.

“Substantially Cytotoxic”, as used herein, refers to a composition that changes the metabolism, proliferation, or viability of mammalian cells that contact the surface of the composition. These may be quantified by the International Standard ISO 10993-5 which defines three main tests to assess the cytotoxicity of materials including the extract test, the direct contact test and the indirect contact test.

“Substantially hemocompatible”, as used herein, means that the composition is substantially non-hemolytic, in addition to being non-thrombogenic and non-immunogenic, as tested by appropriately selected assays for thrombosis, coagulation, and complement activation as described in ISO 10993-4.

“A substantially non-hemolytic surface”, as used herein, means that the composition does not lyse 50%, preferably 20%, more preferably 10%, even more preferably 5%, most preferably 1%, of human red blood cells when the following assay is applied: A stock of 10% washed pooled red blood cells (Rockland Immunochemicals Inc, Gilbertsville, Pa.) is diluted to 0.25% with a hemolysis buffer of 150 mM NaCl and 10 mM Tris at pH 7.0. A 0.5 cm² antimicrobial sample is incubated with 0.75 ml of 0.25% red blood cell suspension for 1 hour at 37° C. The solid sample is removed and cells spun down at 6000 g, the supernatant removed, and the OD414 measured on a spectrophotometer. Total hemolysis is defined by diluting 10% of washed pooled red blood cells to 0.25% in sterile deionized (DI) water and incubating for 1 hour at 37° C., and 0% hemolysis is defined using a suspension of 0.25% red blood cells in hemolysis buffer without a solid sample.

“Non-leaching” or “Substantially non-leaching”, as used herein synonymously, means that the compositions retains >50%, 75%, 90%, 95%, 99% of the immobilized bioactive agent over the course of 7, 14, 30, 90, 365, 1000 days. This can be assessed using radiolabeled active agent followed by implantation in a relevant biological environment.

“Substantially non-toxic”, as used herein, means a surface that is substantially non-hemolytic and substantially non-cytotoxic.

“Tether” or “tethering agent” or “Linker”, as used herein synonymously, refers to any molecule, or set of molecules, or polymer used to covalently immobilize a bioactive agent on a material where the molecule remains as part of the final chemical composition. The tether can be either linear or branched with one or more sites for immobilizing bioactive agents. In one embodiment, the tether is greater than 3 angstroms in length. Optionally, the tether may be non-fouling or a zwitterionic polymer. The tether may be immobilized directly on the substrate or on a polymer, either of which may be non-fouling.

“Zwitterion” or “zwittterionic material” refers to macromolecule, material, or moiety possessing both cationic and anionic groups. In most cases, these charged groups are balanced, resulting in a material with zero net charge. Zwitterionic polymers may include both Polyampholyte (the charged groups on different monomer units) and polybetaine (polymers with the anionic and cationic groups on the same monomer unit). Examples of materials which are not zwitterionic include poly(ethylene glycol).

“Amides” as used herein refers to the amide organic moiety and derivatives thereof.

“Amines” as used herein refers to the amine organic moiety and derivatives thereof.

“Carboxylic Acids” as used herein refers to the carboxylic acid organic moiety and derivatives thereof.

“Sugars” as used herein refers to any monosaccharide, polysaccharide or derivative thereof.

### II. Amino Acids

Synthetic amino acids containing one or more non-fouling groups or moieties have the following chemical formula:

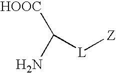

where L is an optional linker group and Z is a non-fouling group including, but not limited to, polyethylene glycol (PEG); oligoethylene glycol (OEG); zwitterionic groups or polymers, such as phosphorycholine, carboxybetaine, and sulfobetaine; groups that are hydrogen bond acceptors but not hydrogen bond donors, such as amides, amide derivatives, amines, amine derivatives, cyclic ethers, sugar derivatives, sulfonates, carboxylic acids, carboxylic acid derivatives, and nitrites; and combinations thereof. Protecting groups including F-moc, Boc, and non-fouling groups may also be attached to the C-terminus, the N-terminus, and/or the linker group. The non-fouling group, Z, may also be protected using protecting groups such as F-moc and Boc. Other suitable non-fouling groups or moieties are disclosed in U.S. Pat. No. 7,276,286 to Chapman et al.

Zwitterionic moieties including phosphorycholines, carboxybetaines, and sulfobetaines are biocompatible and nonfouling groups which are helpful for preparing biocompatible surfaces and materials. Phosphorylcholine is the hydrophilic head group of phospholipids which is the main component of the cellular membrane of red blood cells. The structure of carboxybetaine is similar to that of glycine betaine, which is one of the solutes vital to the osmotic regulation of living organisms. The structure of sulfobetaine is similar to that of 2-aminoethane sulfonic acid or taurine, which is present in high concentrations in animals and occurs in trace amounts in plants. The biomimetic structures of these zwitterionic groups make them nontoxic and biocompatible.

Polymers with zwitterionic moieties have non-fouling properties. Polysulfobetaine methacrylate (SBMA) and polycarboxybetaine methancrylate (CBMA) are non-fouling, as measured by less than 0.3 ng/cm² fibrinogen adsorption. These zwitterionic polymer-grafted surfaces can highly resist nonspecific protein adsorption from plasma and serum, bacterial adhesion, biofilm formation, and platelet adhesion. Polycarboxybetaine polymers also exhibit anticoagulant properties. Most surfaces do not resist platelet attachment, which may sequentially induce thrombosis on the surfaces, unless the fibrinogen adsorption is less than 5-10 ng/cm². Moreover, carboxybetaine polymers have unique dual functionalities—they have abundant functional groups for convenient ligand immobilization and still maintain high resistance to biomolecular attachment.

The linker, L, is typically derived from a molecule having two functional groups capable of forming a covalent bond to another species. Typically, the two functional groups are located at the ends of the linker; however, one or both of the functional groups may be located at a position on the linker other than the ends. The linker can be a single atom, e.g., a sulfur, carbon, oxygen, or nitrogen atom, two or more atoms, such as an amide linker, or as large as an oligomer or polymer. The linker may contain one or more heteroatoms within the linker. Optionally, no linker is used, with the non-fouling group, Z, covalently attached directly to the amino acid backbone. Heterobifunctional crosslinking agents can also be utilized to create the linker. In one embodiment this crosslinking agent could be Sulfo-GMBS. Other possible heterobifunctional crosslinking agents that can be used include, but are not limited to: -[a-maleimidoacetoxy]-succinimide ester (AMAS), N-β-Maleimidopropionic acid (BMPA), N-(R-Maleimidopropionic acid) hydrazide TFA (BMPH), N-R (Maleimidopropyloxy) succinimide ester (BMPS), N-£-Maleimidocaproic acid (EMCA), N-[e-maleimidocaproic acid] hydrazide (EMCH), N-(E-Maleimidocaproyloxy) sulfosuccinimide ester (EMCS), N-K-Maleimidoundecanoic acid (KMUA), N-(K-Maleimidoundecanoic acid) hydrazide (KMUH), LC-SMSS, m-Maleimidobenzoyl-N hydroxysuccinimide ester (MBS), 4-(N-Maleimidomethyl) cyclohexane-1-carboxylhydrazide HCI (M2C2H), 4-(4-N-Maleimidophenyl) butyric acid hydrazide HCI (MPBH), N-succinimidyl S-acetylthioacetate (SATA), N-succinimidyl-S-acetylthiopropionate (SATP), and succinimidyl-4-(N-maleimidomethyl)cyclohexane-1-carboxylate (SMCC).

In one embodiment, the amino acid is a carboxybetaine-based amino acid having the following chemical formula:

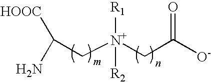

where m=1-5, n=1-5, and R1 and R2=H or C₁₋₃.

### III. Peptides Containing One or More Non-Fouling Amino Acids

A. Non-Fouling Segments

The zwitterionic amino acids can be incorporated into bioactive peptides individually (e.g., randomly) or as block segments. The non-fouling amino acids can be incorporated into the peptides randomly or in a specific sequence. For example, a peptide can be designed with the following structure:

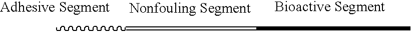

where non-fouling amino acids, such as zwitterionic amino acids, can be polymerized or coupled together to form a non-fouling segment and inserted between an adhesive segment and a bioactive segment. In other embodiments, the adhesive segment may be placed between the non-fouling and bioactive segment, or placed in one of the side chains of the non-fouling or bioactive segment amino acids. In one embodiment, the bioactive segment is an antimicrobial peptide. In another embodiment, the bioactive peptide is a biomarker, adhesion peptide, or other bioactive peptide.

B. Bioactive Segments

The bioactive segment can be designed to accomplish any desired specific interaction at the interface. Bioactive segments include, but are not limited to, adhesion agents such as RGD peptide, serum proteins or portions thereof, growth factors, bone morphogenic proteins, or antimicrobial peptides. Additionally, bioactive segments could be amino acids with side chains that allow the immobilization of secondary agents, such as a heparin for an anti-thrombotic surface.

Antimicrobial peptides (AmPs) are a family of host-defense peptides containing typically between 12 and 100 amino acids. The peptides are generally positively charged and amphiphilic. AmPs can distinguish between mammalian cells and microbes and are highly efficient at killing microbes. It has been shown that AmPs covalently tethered to polymer surfaces retain their bactericidal activity. In order to maximize the ability of AmPs to reach the target bacterial membranes in body fluid (e.g. blood), it would be advantageous to have the AmPs presented on top of or in conjunction with non-fouling groups. Longer activity may be achieved in potentially fouling environments by reducing protein adhesion and binding of materials from dead bacteria that have encountered the surface. Relevant fluids include blood, tears, saliva, hydrocephalus fluid, genital fluids, lymph, and urine, and their derivatives (e.g. serum and plasma).

In one embodiment, peptides with both antimicrobial activity and non fouling properties are prepared by introducing non-fouling unnatural amino acids into antimicrobial sequences. The advantage of this design is no additional non-fouling background or segment is required and the peptides can be directly coated on the surfaces without prior or further treatment of the surfaces. The non-fouling segments can be incorporated into the peptides using non-natural amino acids and thus no conjugation chemistry is needed to covalently bind the AmPs on a non-fouling background.

C. Adhesive Segments

The adhesive segment is any amino acids or peptides which can be attached or immobilized, covalently or non-covalently, to a surface. These adhesive segments may contain one or more amino acids or peptides, such as cysteine, polyhistidine-tag peptides, (3,4-dihydroxyphenylalanine) DOPA-based peptides, and combinations thereof. The adhesive group may be used to tether the peptide, covalently or non-covalently, to a surface. In one embodiment, the thiol in a cysteine is reacted with a maleimide on a surface or with a heterobifunctional crosslinker containing a maleimide such as sulfo-GMBS. Alternately, the adhesive segment is between the antimicrobial segment and the non-fouling amino acid segment. On larger peptides or proteins, the active and non-fouling segments can be alternated. The number of non-natural, non-fouling amino acid repeat units included in the peptide can be tailored to the specific application.

Additionally, the adhesive group may be replaced with a polymerizable group such as a vinyl group so that a brush or comb polymer is formed upon polymerization of the polymerizable group. Each side chain on the comb would be composed of the non-fouling and bioactive segments. The comb may be created from a device surface using ATRP or free radical polymerization, and for some applications it may be preferable that the linkages used are stable in bodily fluids.

Analogous structures may be used to display peptides for sensors or arrays by replacing the antimicrobial segment with a target bonding segment. Free peptides with non-fouling amino acids incorporated could have the non-fouling region segregated at one end, or placed at desired locations throughout the molecule.

D. Peptidomimetics

Peptidomimetics which exhibit activity or properties including, but not limited to, anti antibacterial activity, anti-thrombogenic activity, biomarker activity, non-fouling activity, or adhesive activity, may also be used. Peptidomimetics, as used herein, refers to molecules, which mimic peptide structure. Peptidomimetics have general features analogous to their parent structures, polypeptides, such as amphiphilicity. Examples of such peptidomimetic materials are described in Moore et al., Chem. Rev. 101(12), 3893-4012 (2001). The peptidomimetic materials can be classified into the following categories: α-peptides, β-peptides, γ-peptides, and δ-peptides. Copolymers of these peptides can also be used.

Examples of α-peptide peptidomimetics include, but are not limited to, N,N′-linked oligoureas, oligopyrrolinones, oxazolidin-2-ones, azatides and azapeptides.

Examples of β-peptides include, but are not limited to, β-peptide foldamers, α-aminoxy acids, sulfur-containing β-peptide analogues, and hydrazino peptides.

Examples of γ-peptides include, but are not limited to, γ-peptide foldamers, oligoureas, oligocarbamates, and phosphodiesters.

Examples of δ-peptides include, but are not limited to, alkene-based δ-amino acids and carbopeptoids, such as pyranose-based carbopeptoids and furanose-based carbopeptoids.

Another class of peptidomimetics includes oligomers having backbones which can adopt helical or sheet conformations. Example of such compounds include, but are not limited to, compounds having backbones utilizing bipyridine segments, compounds having backbones utilizing solvophobic interactions, compounds having backbones utilizing side chain interactions, compounds having backbones utilizing hydrogen bonding interactions, and compounds having backbones utilizing metal coordination.

Examples of compounds containing backbones utilizing bipyridine segments include, but are not limited to, oligo(pyridine-pyrimidines), oligo(pyridine-pyrimidines) with hydrazal linkers, and pyridine-pyridazines.

Examples of compounds containing backbones utilizing solvophobic interactions include, but are not limited to, oligoguanidines, aedamers (structures which take advantage of the stacking properties of aromatic electron donor-acceptor interactions of covalently linked subunits) such as oligomers containing 1,4,5,8-naphthalene-tetracarboxylic diimide rings and 1,5-dialkoxynaphthalene rings, and cyclophanes such as substituted N-benzyl phenylpyridinium cyclophanes.

Examples of compounds containing backbones utilizing side chain interactions include, but are not limited to, oligothiophenes such as olihothiophenes with chiral p-phenyl-oxazoline side chains, and oligo(m-phenylene-ethynylene)s.

Examples of compound containing backbones utilizing hydrogen bonding interactions include, but are not limited to, aromatic amide backbones such as oligo(acylated 2,2′-bipyridine-3,3′-diamine)s and oligo(2,5-bis[2-aminophenyl]pyrazine)s, diaminopyridine backbones templated by cyanurate, and phenylene-pyridine-pyrimidine ethynylene backbones templated by isophthalic acid.

Examples of compounds containing backbones utilizing metal coordination include, but are not limited to, zinc bilinones, oligopyridines complexed with Co(II), Co(III), Cu(II), Ni(II), Pd(II), Cr(III), or Y(III), oligo(m-pheylene ethynylene)s containing metal-coordinating cyano groups, and hexapyrrins.

### IV. Pharmaceutical Compositions

The non-fouling amino acids described herein, or peptides containing one or more non-fouling amino acids can be attached to a bioactive agent, particularly proteins or peptides, to increase the stability and/or the half-life of the active agent. The functionalized active agent can be incorporated into a pharmaceutical carrier for enteral or parenteral administration. In a preferred emobidment, the active agent is a protein or peptide, and the route of administration is parenteral.

### V. Methods of Making

A. Non-fouling Amino Acids

Non-fouling amino acids can be synthesized using a variety of methods known in the art. For example, the N-terminus, C-terminus, and/or the side chain of a naturally-occurring or non-naturally occurring amino acid can be derivatized with one or more non-fouling groups or moieties. Alternatively, one or more of the N-terminus, C-terminus, and side chain can be protected with a protecting group to prevent derivitization with the non-fouling group or moiety.

In one embodiment, the non-fouling amino acid is a carboxybetaine-based amino acid prepared in the following manner:

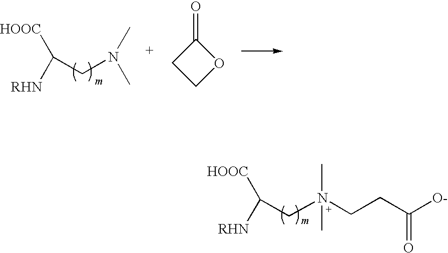

where m=1-5 and the N-terminus of the amino acid can be protected with a protecting group, such as F-moc and Boc.

B. Peptides

These non-natural amino acids may be incorporated into peptides or proteins during chemical synthesis using protected versions of the amino acid including, but not limited to F-moc, Boc, or Z groups. Depending on the chemical nature of the side chain, additional protecting groups may be necessary to block the sidechain from participating in the peptide synthesis reaction. Alternatively, these non-fouling, non-natural amino acids can be incorporated recombinantly by changing the t-RNA assigned to one codon to incorporate the novel amino acid. The altered genetic code can be in vitro translated or used to modify the code of a host production organism, which could be eukaryotic or prokaryotic.

Alternatively, the zwitterionic peptide can be synthesized through a post-reaction of an amino acid or peptide possessing amine groups. In one embodiment, a sulfoboxybetaine peptide can be synthesized from the reaction using an N,N-dimethyl amino group and a propane sultone. In another embodiment, a carboxybetaine peptide can be synthesized from the reaction using an N,N-dimethyl amino group and a propiolactone. In a third embodiment, a carboxybetaine peoptide can be synthesized using a tertiary amino group and a bromoester.

### IV. Methods of Use

The non-fouling amino acids, or peptides containing one or more non-fouling amino acids, can be applied to surfaces, particularly the surfaces of medical devices, in order to improve biocompatibility, reduce thrombogenesis (such as on the surface of stents), prevent infections, and/or reduce fouling by proteins or bacteria present in solution. This is particularly applicable for surfaces where immobilized proteins and peptides are present because non-specific protein or cell fouling may cover these immobilized molecules. Immobilized protein or peptide surfaces may be used in arrays/sensors, or to create antimicrobial surfaces using antimicrobial peptides. In addition, non-fouling groups may be incorporated into molecules in solution to improve stability or half-life in the blood stream. This is particularly applicable to peptides and proteins whose circulation time may be improved through addition of non-fouling, shielding groups, or for drug/gene delivery materials.

Suitable devices include, but are not limited to, surgical, medical or dental instruments, ophthalmic devices, wound treatments (bandages, sutures, cell scaffolds, bone cements, particles), appliances, implants, scaffolding, suturing material, valves, pacemaker, stents, catheters, rods, implants, fracture fixation devices, pumps, tubing, wiring, electrodes, contraceptive devices, feminine hygiene products, endoscopes, wound dressings and other devices, which come into contact with tissue, cells or bodily fluids.

A. Fibrous and Particulate Materials

In one embodiment, the peptides having biological activity, such as antimicrobial activity, are applied to a fibrous material, or are incorporated into a fibrous material or a coating on a fibrous material. These include wound dressings, bandages, gauze, tape, pads, sponges, including woven and non-woven sponges and those designed specifically for dental or ophthalmic surgeries (See, e.g., U.S. Pat. Nos. 4,098,728; 4,211,227; 4,636,208; 5,180,375; and 6,711,879), paper or polymeric materials used as surgical drapes or clothing, disposable diapers, tapes, bandages, feminine products, dressings, bandages, cell scaffolds, sutures, and other fibrous materials. One of the advantages of the immobilized antimicrobial agents is that they are not only antibacterial at the time of application, but help to minimize contamination by the materials after disposal.

Fibrous materials are also useful in cell culture and tissue engineering devices. Bacterial and fungal contamination is a major problem in eukaryotic cell culture and this provides a safe and effective way to minimize or eliminate contamination of the cultures.

The antimicrobial agents are also readily bound to particles, including nanoparticles, microparticles and millimeter beads, which have uses in a variety of applications including cell culture and drug delivery.

B. Implanted and Inserted Materials

The bioactive peptides can also be applied directly to, and coupled by ionic, covalent or hydrogen bonding to, or incorporated into, polymeric, metallic, or ceramic substrates. Suitable devices include, but are not limited to surgical, medical or dental instruments, blood oxygenators, pumps, tubing, wiring, electrodes, contraceptive devices, endoscopes, grafts, stents, pacemakers, implantable cardioverter-defibrillators, cardiac resynchronization therapy devices, ventricular assist devices, heart valves, catheters (including vascular, urinary, neurological, peritoneal, interventional, etc.), shunts, wound drains, dialysis membranes, infusion ports, cochlear implants, endotracheal tubes, guide wires, fluid collection bags, sensors, bone cements, ophthalmic devices, orthopedic devices (hip implants, knee implants, spinal implants, screws, plates, rivets, rods, intramedullary nails, bone cements, artificial tendons, and other prosthetics or fracture repair devices), dental implants, breast implants, penile implants, maxillofacial implants, cosmetic implants, valves, appliances, needles, hernia repair meshes, tension-fee vaginal tape and vaginal slings, tissue regeneration or cell culture devices, or other medical devices used within or in contact with the body or any portion of any of these. Studies demonstrate that high loading can be achieved by direct coupling of antimicrobial agents to polyurethane and silicone, primary materials used for devices such as CVCs. Preferably, the antimicrobial coating herein does not significantly adversely affect the desired physical properties of the device including, but not limited to, flexibility, durability, kink resistance, abrasion resistance, thermal and electrical conductivity, tensile strength, hardness, burst pressure, etc.

C. Coatings, Paints, Dips, Sprays

The antimicrobial agents can also be added to solutions, suspensions, dispersions, paints and other coatings and filters to prevent mildew, bacterial contamination, and in other applications where it is desirable to provide antimicrobial activity.

The present invention will be further understood by reference to the following non-limiting examples.

### EXAMPLES

### Example 1

### Proposed Synthesis of Carboxybetaine Amino Acid

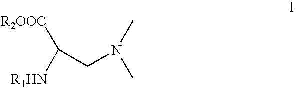

A carboxybetaine amino acid will be synthesized by the reaction of compound 1 (R1=Formoc (9H-(f)luoren-9-yl(m)eth(o)xy(c)arbonyl group) and R2=tert-Butyl group) and β-propiolactone. β-Propiolactone (12 mmol) in 10 mL dried acetone will be added dropwise to a solution of compound 1 (10 mmol) dissolved in 50 mL anhydrous acetone. The reaction mixture will be stirred under nitrogen protection at 15° C. for about 5 h. The white precipitate will be washed with 50 mL anhydrous acetone and 100 mL anhydrous ether. The product will be dried under reduced pressure to obtain the final product. The product will be kept in a dessicator at 2-8° C. before deprotection and polymerization.

### Example 2

### Proposed Synthesis of Peptides Containing Zwitterionic Pendant Groups

Peptide containing pendant groups N,N-dimethyl tertiary amino will be synthesized by solid Fmoc solid-phase peptide synthesis. The peptide (with 50 mmol equiv N,N-dimethyl tertiary amino groups), ethyl 6-bromohexanoate (55 mmol), and acetonitrile (25 mL), will be added into a 100-mL round-bottom flask. The mixture will be stirred under a nitrogen atmosphere for five days at 45° C. The solvent will be removed on a rotary evaporator under reduced pressure. The peptide will be purified by dialysis for several times using deionized water. After dialysis, the peptide will be lyophilized and dried under reduced pressure. The peptide will then be hydrolyzed in a mixed solvent of methanol and water (1:1 ratio) and NaOH (1 M) for 2 hours at room temperature. The products will be dialyzed and lyophilized.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A synthetic amino acid having Formula I 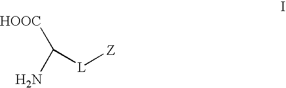
where L is an optional linker group and Z is a non-fouling group.

2. The amino acid of claim 1, wherein Z is selected from the group consisting of phosphorycholine, carboxybetaine, sulfobetaine, intramolecular zwitterionic groups, or a group that is a hydrogen bond acceptor but not a hydrogen bond donor.

3. The amino acid of claim 2, wherein the group that is a hydrogen bond acceptor, but not a hydrogen bond donor, is selected from the group consisting of amides, amines, cyclic ethers, sugars, sulfonates, carboxylic acids, nitrites, and combinations thereof.

4. The amino acid of claim 2, having the Formula II: 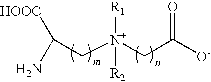
where m=1-5, n=1-5, R1, R2=H or C₁₋₃.

5. The amino acid of claim 1 wherein the linking group, L, remains intact for a period of no more than 30 days in the bloodstream in vivo.

6. The amino acid of claim 1 wherein the non-fouling side chain Z retains antifouling activity after 30 days in the bloodstream in vivo.

7. The amino acid of claim 1 wherein the amino acid is a non-natural or D-amino acid.

8. The amino acid of claim 1 incorporated into a peptide or protein.

9. A peptide comprising a bioactive segment and a non-fouling segment, wherein the non-fouling segment comprises one or more amino acids of claim 1.

10. The peptide of claim 9, wherein Z is selected from the group consisting of phosphorycholine, carboxybetaine, sulfobetaine, intramolecular zwitterionic groups, or a group that is a hydrogen bond acceptor but not a hydrogen bond donor.

11. The peptide of claim 10, wherein the group that is a hydrogen bond acceptor, but not a hydrogen bond donor, is selected from the group consisting of amides, amines, cyclic ethers, sugars, sulfonates, carboxylic acids, nitriles, and combinations thereof.

12. The peptide of claim 9, wherein the amino acid has Formula II: 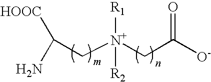
where m=1-5, n=1-5, R1, R2=H or C₁₋₃.

13. The peptide of claim 9 wherein the linking group, L, remains intact for a period of no more than 30 days in the bloodstream in vivo.

14. The peptide of claim 9 wherein the non-fouling side chain Z retains antifouling activity after 30 days in the bloodstream in vivo.

15. The peptide of claim 1 wherein the amino acid is a non-natural or D-amino acid.

16. The peptide of claim 9, wherein the bioactive segment is selected from the group consisting of therapeutic, prophylactic and diagnostic peptides.

17. The peptide of claim 9, wherein the bioactive segment is an antimicrobial segment, a biomarker, a cell adhesion peptide, or a peptide.

18. The peptide of claim 9, further comprising an adhesive segment.

19. The peptide of claim 18, wherein the adhesive segment is selected from the group consisting of cysteine, polyhistidine-tag peptides, and 3,4-dihydroxyphenylalanine (DOPA)-based peptides.

20. The peptide of claim 18, wherein the adhesive segment is capable of tethering the peptide covalently or non-covalently to a surface.

21. The peptide of claim 9, wherein one end of the non-fouling segment is bound to the bioactive segment and the other end of the non-fouling segment is bound to the adhesive segment.

22. The peptide of claim 18, wherein one end of the adhesive segment is bound to the bioactive segment and the other end of the adhesive segment is bound to the non-fouling segment.

23. The peptide of claim 9, wherein the peptide further comprises a polymerizable group.

24. The peptide of claim 23, wherein the polymerizable group is a vinyl group.

25. A pharmaceutical composition comprising the peptide of claim 9.

26. The composition of claim 25, wherein Z in the peptide is selected from the group consisting of phosphorycholine, carboxybetaine, sulfobetaine, intramolecular zwitterionic groups, or a group that is a hydrogen bond acceptor but not a hydrogen bond donor.

27. The composition of claim 26, wherein the group that is a hydrogen bond acceptor, but not a hydrogen bond donor, is selected from the group consisting of amides, amines, cyclic ethers, sugars, sulfonates, carboxylic acids, nitrites, and combinations thereof.

28. The composition of claim 25, wherein the peptide comprises one or amino acids of Formula II: 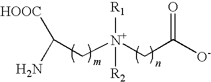
where m=1-5, n=1-5, R1, R2=H or C₁₋₃.

29. The composition of claim 25 wherein the linking group, L, in the amino acid remains intact for a period of no more than 30 days in the bloodstream in vivo.

30. The composition of claim 25 wherein the non-fouling side chain Z in the amino acid retains antifouling activity after 30 days in the bloodstream in vivo.

31. The composition of claim 25 wherein the amino acid is a non-natural or D-amino acid.

32. The composition of claim 25, wherein the bioactive segment is selected from the group consisting of therapeutic, prophylactic and diagnostic peptides.

33. The composition of claim 25, wherein the bioactive segment is an antimicrobial segment, a biomarker, a cell adhesion peptide, or a peptide.

34. The composition of claim 25, further comprising an adhesive segment.

35. The composition of claim 34, wherein the adhesive segment is selected from the group consisting of cysteine, polyhistidine-tag peptides, and 3,4-dihydroxyphenylalanine (DOPA)-based peptides.

36. The composition of claim 34, wherein the adhesive segment is capable of tethering the peptide covalently or non-covalently to a surface.

37. The composition of claim 34, wherein one end of the non-fouling segment is bound to the bioactive segment and the other end of the non-fouling segment is bound to the adhesive segment.

38. The composition of claim 34, wherein one end of the adhesive segment is bound to the bioactive segment and the other end of the adhesive segment is bound to the non-fouling segment.

39. The composition of claim 25, wherein the peptide further comprises a polymerizable group.

40. The composition of claim 39, wherein the polymerizable group is a vinyl group.

41. A composition or device comprising a substrate having immobilized thereon or therein one or more peptides of claim 9,

42. The composition or device of claim 41, wherein the peptide comprises an antimicrobial peptide.

43. The composition or device of claim 41, wherein the peptides are immobilized by bonds selected from the group consisting of covalent bonds, non-covalent bonds, and combinations of covalent and non-covalent bonds thereof.

44. The composition of claim 41, wherein the substrate is formed from a material selected from the group consisting of polymeric materials, metallic materials, and ceramic materials.

45. The composition of claim 44, wherein the substrate is the surface of a medical device.

46. The composition of claim 45, wherein the medical device is selected from the group consisting of surgical, medical or dental instruments, ophthalmic devices, wound treatments, bandages, sutures, cell scaffolds, bone cements, particles, appliances, implants, scaffolding, suturing material, valves, pacemaker, stents, catheters, rods, implants, fracture fixation devices, pumps, tubing, wiring, electrodes, contraceptive devices, feminine hygiene products, endoscopes, wound dressings and other devices, which come into contact with tissue.

47. An isolated synthetic amino acid having Formula I 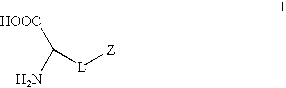
where L is an optional linker group and Z is polyethylene glycol (PEG) or an oligoethylene glycol (OEG) and the amino acid is natural or non-natural, L or D.

48. A protected peptide having Formula III 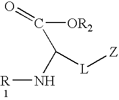
wherein
L is an optional linker group,
Z is a non-fouling group,
R₁ is selected from the group consisting of a 9-fluorenylmethyl chloroformate (F-moc) group, a t-butyl carbamate (Boc) group, or a benzyloxycarbonyl group, and
R₂ is selected from the group consisting of hydrogen, methyl, benzyl, t-butyl, and silyl.

49. A method of making the amino acid of claim 1, the method comprising derivatizing the N-terminus, C-terminus, and/or side chain of a naturally occurring or non-naturally occurring amino acid with one or more non-fouling groups or moieties.

50. The method of claim 49, wherein the non-fouling group is a zwitterionic group.

51. A method of making the amino acid of claim 48, the method comprising protecting the amino group, carboxylic acid group, a side chain, or combinations thereof with a protecting group and derivatizing the unprotected amino group, carboxylic acid group, side chain, or combinations thereof with one or more non-fouling groups or moieties.

52. The method of claim 51, wherein the non-fouling group is a zwitterionic group.

53. A method of making a peptide comprising an amino acid of claim 1, the method comprising incorporating a protected or unprotected amino acid of claim 1 into a peptide.

54. The method of claim 53, wherein the peptide is prepared by chemical synthesis.

55. The method of claim 53, wherein the peptide is prepared recombinantly.

56. A method of making a peptide comprising an amino acid of claim 1, the method comprising incorporating a naturally occurring or non-naturally occurring amino acid into a peptide and derivatizing one or more amino acids in the peptide with a non-fouling group or moiety.

57. The method of claim 56, wherein the non-fouling moiety is a zwitterionic group.
